(19) United States
(12) Patent Application Publication
(10) Pub. No.: US 2026/0240525 A1
(43) Pub. Date: Aug. 20, 2026

(54) SYSTEM FOR STRAIN IMAGING IN CONTRAST ECHOCARDIOGRAPHY

(71) Applicant: GE Precision Healthcare LLC, Waukesha, WI (US)

(72) Inventor: Hani Nozari MIRAR, Oslo (NO)

(21) Appl. No.: 19/057,546

(22) Filed: Feb. 19, 2025

(51) Int. Cl. A61B 8/00; A61B 8/08

(52) U.S. Cl. CPC A61B 8/485; A61B 8/0883; A61B 8/463; A61B 8/481

(57) ABSTRACT

A system may receive contrast-enhanced ultrasound data of an anatomical structure of a heart of a subject during a first cardiac cycle of the heart of the subject, and receive non-enhanced ultrasound data of the anatomical structure of the heart of the subject during a second cardiac cycle of the heart of the subject. The system may determine a first region of interest (ROI) of the anatomical structure in the contrast-enhanced ultrasound data, and determine a second ROI of the anatomical structure in the non-enhanced ultrasound data based on the first ROI of the anatomical structure in the contrast-enhanced ultrasound data. The system may perform joint strain imaging using the first ROI in the contrast-enhanced ultrasound data and the second ROI in the non-enhanced ultrasound data. The system may determine and display information related to cardiac deformation of the anatomical structure of the heart of the subject.

### TECHNICAL FIELD

The present disclosure relates, generally, to a system for performing joint strain imaging using contrast-enhanced ultrasound data and non-enhanced ultrasound data. More specifically, the present disclosure relates to a system that receives contrast-enhanced ultrasound data of an anatomical structure of a heart of a subject corresponding to a first cardiac cycle of the heart of the subject, receives non-enhanced ultrasound data of the anatomical structure of the heart of the subject corresponding to a second cardiac cycle of the heart of the subject, determines a region of interest (ROI) of the anatomical structure of the heart of the subject using the contrast-enhanced ultrasound data, and performs joint strain imaging using the ROI with respect to the contrast-enhanced ultrasound data and the non-enhanced ultrasound data.

### BACKGROUND

Strain imaging may refer to an imaging technique to evaluate myocardial deformation. Strain may refer to the change in cardiac length of the myocardium from end-diastole to end-systole. Strain imaging may be used for assessment of myocardial mechanics. For example, strain imaging may be used to detect cardiomyopathies, heart disease, myocardial dysfunction, or the like. For strain imaging using ultrasound data, an ROI of the heart may be segmented into a set of myocardial segments in the ultrasound data. The myocardial segments may be tracked during the cardiac cycle using a strain imaging technique (e.g., a template matching technique, an image registration technique, an artificial intelligence (AI) technique, or the like) and the ultrasound data. Strain values for the set of myocardial segments may be determined based on the tracking of the myocardial segments. For example, a strain curve, or “strain trace,” that includes strain values for a myocardial segment over the cardiac cycle may be determined. Various relevant strain values (e.g., end-systolic strain, peak systolic strain, peak strain, etc.) may be determined from the strain curve.

Contrast echocardiography, which may also be referred to as “contrast-enhanced ultrasound,” is an ultrasound imaging technique that utilizes an acoustically active contrast medium (e.g., microbubbles) that circulate through the cardiovascular system during ultrasound imaging. The contrast medium creates a highly-reflective ultrasound image. In this way, contrast-enhanced ultrasound is particularly useful to enhance visualization of the endocardial borders. Contrast-enhanced ultrasound may involve pulse inversion techniques that enhance contrast resolution by transmitting pairs of ultrasound signals with opposite phases, and combining received echo signals to cancel out tissue signals while enhancing signals from contrast agents. This technique improves delineation of the endocardial border. In this way, contrast-enhanced ultrasound may be beneficial for strain imaging because, as compared to non-enhanced echocardiography (e.g., B-mode echocardiography), contrast-enhanced ultrasound provides a more accurate estimate of myocardium length, especially for patients with hypertrophic cardiomyopathy.

However, contrast-enhanced ultrasound may provide a very weak tissue signal, provide a drop in acquisition frame rate, and provide reduced visibility of the mitral valve hinge points. The foregoing may all inhibit the accuracy and efficacy of strain imaging using contrast-enhanced ultrasound. For instance, due to the specific imaging setup for contrast-enhanced ultrasound, the frame rate typically drops below the minimum required frame rate for strain imaging. Further, contrast-enhanced ultrasound in strain imaging might not create discernible features in the basal territories that persist throughout the cardiac cycle. This may render it difficult to distinguish between tissue and cavity, especially when the myocardium is thin. This adversely affects the accuracy of strain imaging. Basal points may be critical landmarks in strain imaging and serve as the primary drivers of longitudinal strain. If the tracking of these basal points fails, the global longitudinal strain may be inaccurate. In non-enhanced ultrasound, these regions are robust features to track during strain imaging. Further still, contrast echo signals may inhibit tissue signals. Therefore, it might not be possible to define and track segmental longitudinal borders using contrast-enhanced ultrasound.

Non-enhanced ultrasound may underestimate left ventricular volumes. In contrast, ultrasound-enhancing agents may improve accuracy by providing clearer visualization of endocardial borders, which may aligning measurements more closely with cardiac magnetic resonance imaging. Further, contrast-enhanced ultrasound improves the accuracy and reproducibility of global strain measurements, which may result in better agreement with cardiac magnetic resonance (CMR), even in patients with suboptimal acoustic windows.

Strain imaging algorithms may struggle to track various structures in contrast-enhanced ultrasound data because the structures may lack discernible features. When an ROI is placed in this area, the ROI typically drops down to the left atrium (LA) or the left ventricle (LV) cavities during strain imaging, which may result in incorrect global longitudinal strain.

To accurately report segmental strain on contrast-enhanced ultrasound images, it might be important to effectively track the longitudinal segmental borders over time. However, due to the very low tissue signal intensity, tracking these borders becomes challenging using contrast-enhanced ultrasound and renders it difficult to report an accurate segmental strain.

In light of the foregoing, non-enhanced ultrasound might not provide a clear delineation of the myocardium, but may offer a high framerate. Further, non-enhanced ultrasound provides robust acoustic markers that are viable for strain imaging. Contrast-enhanced ultrasound suffers from low framerate and does not provide strong markers to track tissue. However, contrast-enhanced ultrasound offers highly accurate myocardial border definition with correct myocardial layer lengths.

### SUMMARY

This summary introduces concepts that are described in more detail in the detailed description. It should not be used to identify essential features of the claimed subject matter, nor to limit the scope of the claimed subject matter.

In an aspect, a system may include a lens, an acoustic matching layer, an acoustic dematching layer, and a plurality of transducer elements; a memory configured to store instructions; and one or more processors configured to execute the instructions to: receive contrast-enhanced ultrasound data of an anatomical structure of a heart of a subject during a first cardiac cycle of the heart of the subject; receive non-enhanced ultrasound data of the anatomical structure of the heart of the subject during a second cardiac cycle of the heart of the subject; determine a first region of interest (ROI) of the anatomical structure of the heart of the subject in the contrast-enhanced ultrasound data; determine a second ROI of the anatomical structure of the heart of the subject in the non-enhanced ultrasound data based on the first ROI of the anatomical structure of the heart of the subject in the contrast-enhanced ultrasound data; perform joint strain imaging using the first ROI of the anatomical structure of the heart of the subject in the contrast-enhanced ultrasound data and the second ROI of the anatomical structure of the heart of the subject in the non-enhanced ultrasound data; determine information related to cardiac deformation of the anatomical structure of the heart of the subject based on performing the joint strain imaging; and display the information related to cardiac deformation of the anatomical structure of the heart of the subject.

In another aspect, a method may include receiving contrast-enhanced ultrasound data of an anatomical structure of a heart of a subject during a first cardiac cycle of the heart of the subject; receiving non-enhanced ultrasound data of the anatomical structure of the heart of the subject during a second cardiac cycle of the heart of the subject; determining a first region of interest (ROI) of the anatomical structure of the heart of the subject in the contrast-enhanced ultrasound data; determining a second ROI of the anatomical structure of the heart of the subject in the non-enhanced ultrasound data based on the first ROI of the anatomical structure of the heart of the subject in the contrast-enhanced ultrasound data; performing joint strain imaging using the first ROI of the anatomical structure of the heart of the subject in the contrast-enhanced ultrasound data and the second ROI of the anatomical structure of the heart of the subject in the non-enhanced ultrasound data; determining information related to cardiac deformation of the anatomical structure of the heart of the subject based on performing the joint strain imaging; and displaying the information related to cardiac deformation of the anatomical structure of the heart of the subject.

In yet another aspect, a non-transitory computer-readable medium may store instructions that, when executed by one or more processors, cause the one or more processors to: receive contrast-enhanced ultrasound data of an anatomical structure of a heart of a subject during a first cardiac cycle of the heart of the subject; receive non-enhanced ultrasound data of the anatomical structure of the heart of the subject during a second cardiac cycle of the heart of the subject; determine a first region of interest (ROI) of the anatomical structure of the heart of the subject in the contrast-enhanced ultrasound data; determine a second ROI of the anatomical structure of the heart of the subject in the non-enhanced ultrasound data based on the first ROI of the anatomical structure of the heart of the subject in the contrast-enhanced ultrasound data; perform joint strain imaging using the first ROI of the anatomical structure of the heart of the subject in the contrast-enhanced ultrasound data and the second ROI of the anatomical structure of the heart of the subject in the non-enhanced ultrasound data; determine information related to cardiac deformation of the anatomical structure of the heart of the subject based on performing the joint strain imaging; and display the information related to cardiac deformation of the anatomical structure of the heart of the subject.

### BRIEF DESCRIPTION OF DRAWINGS

FIG. 1 is a diagram of an example system for performing joint strain imaging using contrast-enhanced ultrasound data and non-enhanced ultrasound data.

FIG. 2 is a diagram of an example strain imaging system for performing joint strain imaging using contrast-enhanced ultrasound data and non-enhanced ultrasound data.

FIG. 3 is a diagram of an example ultrasound system for acquiring contrast-enhanced ultrasound data and non-enhanced ultrasound data.

FIG. 4 is a diagram of an example preoperative imaging system for receiving preoperative imaging data of the region of interest of the anatomical feature of the heart of the subject.

FIG. 5 is a flowchart of an example process for performing joint strain imaging using contrast-enhanced ultrasound data and non-enhanced ultrasound data.

FIG. 6 is a diagram of contrast-enhanced ultrasound data and non-enhanced ultrasound data.

FIG. 7 is a diagram of a first ROI in contrast-enhanced ultrasound data and a second ROI in non-enhanced ultrasound data.

FIG. 8 is a diagram of tracking points in the contrast-enhanced ultrasound data and the non-enhanced ultrasound data.

FIG. 9 is a diagram of an example user interface for displaying the information related to cardiac deformation of the anatomical structure of the heart of the subject.

FIG. 10 is a diagram of an example user interface for displaying the information related to cardiac deformation of the anatomical structure of the heart of the subject.

### DETAILED DESCRIPTION

As addressed above, strain imaging may be performed using non-enhanced ultrasound data or contrast-enhanced ultrasound data. Each type of ultrasound data has various benefits and drawbacks for performing strain imaging. For instance, non-enhanced ultrasound data may offer a high framerate and may provide robust acoustic markers that are viable for strain imaging, but might not provide a clear delineation of the myocardium. Contrast-enhanced ultrasound data may offer highly accurate myocardial border definition with correct myocardial layer lengths, but might not provide high framerate and might not provide strong markers to track tissue.

The present disclosure provides an image acquisition mode that operates over multiple complete cardiac cycles after contrast agent injection. A cardiac cycle utilizes contrast-enhanced ultrasound (e.g., a contrast-specific imaging setting), and a subsequent cardiac cycle uses non-enhanced ultrasound (e.g., a B-mode imaging setting). The contrast-enhanced ultrasound may yield a clear delineation of the myocardial borders and length. The non-enhanced ultrasound provides a high framerate scan with robust acoustic markers for strain imaging, along with well-defined longitudinal segmental borders. An ROI can be automatically extracted from the cardiac cycle associated with the contrast-enhanced ultrasound. The ROI can be used to perform joint strain imaging.

By utilizing both contrast-enhanced ultrasound data and non-enhanced ultrasound data and performing joint strain imaging, the embodiments herein provide an improvement in the technical field of strain imaging by more accurately determining information related to cardiac deformation of an anatomical structure of a heart of a subject. Further, in this way, some embodiments herein provide an improvement to strain imaging systems by permitting the strain imaging systems to more accurately determine information related to cardiac deformation of an anatomical structure of a heart of a subject.

More specifically, according to an embodiment, a system may receive contrast-enhanced ultrasound data of an anatomical structure of a heart of a subject during a first cardiac cycle of the heart of the subject; receive ultrasound data of the anatomical structure of the heart of the subject during a second cardiac cycle of the heart of the subject; determine a first region of interest (ROI) of the anatomical structure of the heart of the subject in the contrast-enhanced ultrasound data; determine a second ROI of the anatomical structure of the heart of the subject in the ultrasound data based on the first ROI of the anatomical structure of the heart of the subject in the contrast-enhanced ultrasound data; perform joint strain imaging using the first ROI of the anatomical structure of the heart of the subject in the contrast-enhanced ultrasound data and the second ROI of the anatomical structure of the heart of the subject in the ultrasound data; determine information related to cardiac deformation of the anatomical structure of the heart of the subject based on performing the joint strain imaging; and display the information related to cardiac deformation of the anatomical structure of the heart of the subject.

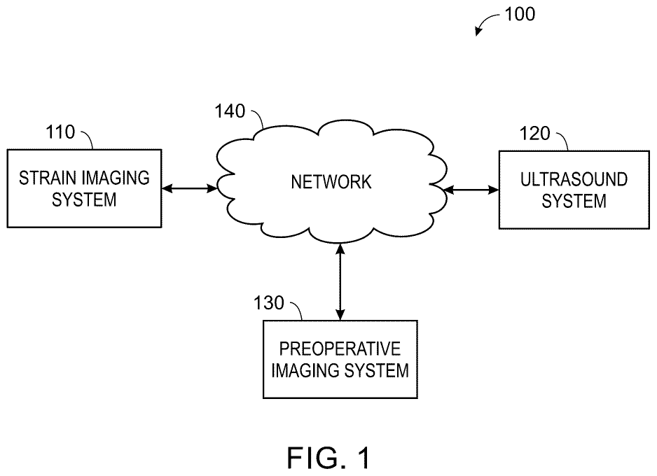

FIG. 1 is a diagram of an example system for performing joint strain imaging using contrast-enhanced ultrasound data and non-enhanced ultrasound data. As shown in FIG. 1, the system 100 may include a strain imaging system 110, an ultrasound system 120, a preoperative imaging system 130, and a network 140.

The strain imaging system 110 may be configured to receive contrast-enhanced ultrasound data of an anatomical structure of a heart of a subject during a first cardiac cycle of the heart of the subject; receive ultrasound data of the anatomical structure of the heart of the subject during a second cardiac cycle of the heart of the subject; determine a first ROI of the anatomical structure of the heart of the subject in the contrast-enhanced ultrasound data; determine a second ROI of the anatomical structure of the heart of the subject in the ultrasound data based on the first ROI of the anatomical structure of the heart of the subject in the contrast-enhanced ultrasound data; perform joint strain imaging using the first ROI of the anatomical structure of the heart of the subject in the contrast-enhanced ultrasound data and the second ROI of the anatomical structure of the heart of the subject in the ultrasound data; determine information related to cardiac deformation of the anatomical structure of the heart of the subject based on performing the joint strain imaging; and display the information related to cardiac deformation of the anatomical structure of the heart of the subject. For example, the strain imaging system 110 may be a computer, a server, a medical device, or the like.

The ultrasound system 120 may be configured to acquire contrast-enhanced ultrasound data of an anatomical feature of a heart of a subject, and may be configured to acquire non-enhanced ultrasound data of the anatomical feature of the heart of the subject. For example, the ultrasound system 120 may be a two-dimensional (2D) ultrasound system, a three-dimensional (3D) ultrasound system, a four-dimensional (4D) ultrasound system, a Doppler ultrasound system, or the like. The subject may be a person, an animal, a phantom, or the like.

The preoperative imaging system 130 may be configured to receive preoperative imaging data of the region of interest of the heart of the subject. For example, the preoperative imaging system 130 may be a computed tomography (CT) system, a magnetic resonance imaging (MRI) system, an ultrasound system, an X-ray system, a positron emission tomography (PET) device, or the like.

The network 140 may permit communication between the strain imaging system 110, the ultrasound system 120, and the preoperative imaging system 130. For example, the network 140 may be a local area network (LAN), a wide area network (WAN), a metropolitan area network (MAN), a cellular network, a private network, an ad hoc network, an intranet, the Internet, a fiber optic-based network, a wired network, a wireless network, or the like, and/or a combination of these or other types of networks.

The number and arrangement of the system 100 are provided as an example. In practice, the system 100 may include additional systems, fewer systems, different systems, or differently arranged systems than those shown in FIG. 1. Additionally, or alternatively, a set of systems (e.g., one or more systems) of the system 100 may be integrated into a single system, and/or perform one or more functions described as being performed by another system, or set of systems, of the system 100.

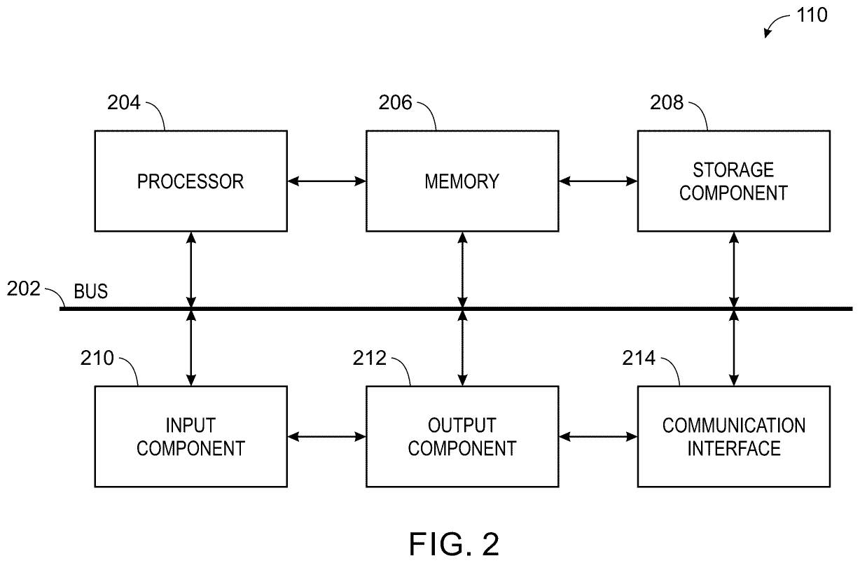

FIG. 2 is a diagram of an example strain imaging system for performing joint strain imaging using contrast-enhanced ultrasound data and non-enhanced ultrasound data. As shown in FIG. 2, the strain imaging system 110 may include a bus 202, a processor 204, a memory 206, a storage component 208, an input component 210, an output component 212, and a communication interface 214.

The bus 202 includes a component that permits communication among the components of the strain imaging system 110. The processor 204 may be implemented in hardware, firmware, or a combination of hardware and software. The processor 204 may be a central processing unit (CPU), a graphics processing unit (GPU), an accelerated processing unit (APU), a microprocessor, a microcontroller, a digital signal processor (DSP), a field-programmable gate array (FPGA), an application-specific integrated circuit (ASIC), or another type of processing component.

The processor 204 may include one or more processors capable of being programmed to perform a function. The processor 204 may include one or more processors 204 configured to perform the operations described herein. For example, a single processor 204 may be configured to perform all of the operations described herein. Alternatively, multiple processors 204, collectively, may be configured to perform all of the operations described herein, and each of the multiple processors 204 may be configured to perform a subset of the operations described herein. For example, a first processor 204 may perform a first subset of the operations described herein, a second processor 204 may be configured to perform a second subset of the operations described herein, etc.

The memory 206 may include a random access memory (RAM), a read only memory (ROM), and/or another type of dynamic or static storage device (e.g., a flash memory, a magnetic memory, and/or an optical memory) that stores information and/or instructions for use by the processor 204.

The storage component 208 may store information and/or software related to the operation and use of the strain imaging system 110. For example, the storage component 208 may include a hard disk (e.g., a magnetic disk, an optical disk, a magneto-optic disk, and/or a solid state disk), a compact disc (CD), a digital versatile disc (DVD), a floppy disk, a cartridge, a magnetic tape, and/or another type of non-transitory computer-readable medium, along with a corresponding drive.

The input component 210 may include a component that permits the strain imaging system 110 to receive information, such as via user input (e.g., a touch screen display, a keyboard, a keypad, a mouse, a button, a switch, a camera, and/or a microphone). Additionally, or alternatively, the input component 210 may include a sensor for sensing information (e.g., a global positioning system (GPS) component, an accelerometer, a gyroscope, and/or an actuator). The output component 212 may include a component that provides output information from the strain imaging system 110 (e.g., a display, a speaker for outputting sound at the output sound level, and/or one or more light-emitting diodes (LEDs)).

The communication interface 214 may include a transceiver-like component (e.g., a transceiver and/or a separate receiver and transmitter) that enables the strain imaging system 110 to communicate with other systems, such as via a wired connection, a wireless connection, or a combination of wired and wireless connections. The communication interface 214 may permit the strain imaging system 110 to receive information from another system and/or provide information to another system. For example, the communication interface 214 may include an Ethernet interface, an optical interface, a coaxial interface, an infrared interface, a radio frequency (RF) interface, a universal serial bus (USB) interface, a Wi-Fi interface, a cellular network interface, or the like.

The strain imaging system 110 may perform one or more processes described herein. The strain imaging system 110 may perform these processes based on the processor 204 executing software instructions stored by a non-transitory computer-readable medium, such as the memory 206 and/or the storage component 208. A computer-readable medium may be defined herein as a non-transitory memory device. A memory device may include memory space within a single physical storage device or memory space spread across multiple physical storage devices.

The software instructions may be read into the memory 206 and/or the storage component 208 from another computer-readable medium or from another system via the communication interface 214. When executed, the software instructions stored in the memory 206 and/or the storage component 208 may cause the processor 204 to perform one or more processes described herein. Additionally, or alternatively, hardwired circuitry may be used in place of or in combination with software instructions to perform one or more processes described herein. Thus, implementations described herein are not limited to any specific combination of hardware circuitry and software.

The number and arrangement of the components shown in FIG. 2 are provided as an example. In practice, the strain imaging system 110 may include additional components, fewer components, different components, or differently arranged components than those shown in FIG. 2. Additionally, or alternatively, a set of components (e.g., one or more components) of the strain imaging system 110 may perform one or more functions described as being performed by another set of components of the strain imaging system 110.

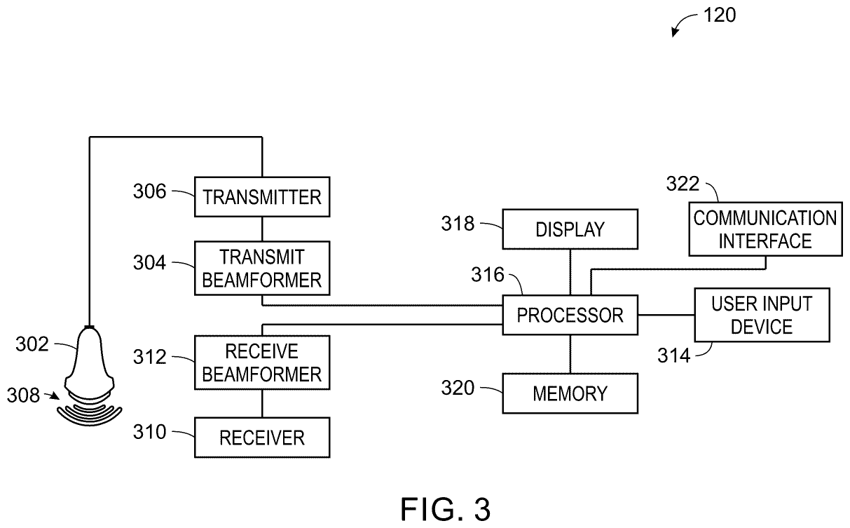

FIG. 3 is a diagram of an example ultrasound system for acquiring contrast-enhanced ultrasound data and non-enhanced of the region of interest of the heart of the subject. As shown in FIG. 3, the ultrasound system 120 may include an ultrasound probe 302, a transmit beamformer 304, a transmitter 306, elements 308 a receiver 310, a receive beamformer 312, a user input device 314, a processor 316, a display 318, a memory 320, and a communication interface 322. The foregoing components may be connected via wired or wireless connections.

The ultrasound probe 302 may be configured to receive ultrasound data. For example, the ultrasound probe 302 may be a linear probe, a phase array probe, a curved linear probe coupled with a position tracking system, a mechanically steered linear array transducer, a phased array transducer, a curved linear array transducer, an electronically steered 2D transducer array, an electronic 3D (e3D) probe, an electronic 4d (e4D) probe, a low profile wearable patch version of any of the foregoing probes, or the like. According to an embodiment, the ultrasound probe 302 may be configured to generate ultrasound signals, emit the ultrasound signals towards the region of interest of a subject, receive echo ultrasound signals that are back-scattered from the region of interest of the subject, generate ultrasound data based on the echo ultrasound signals, and output the ultrasound data. The ultrasound probe 302 may include a lens, an acoustic matching layer, the transducer elements 308, an acoustic dematching layer, and a backing layer.

According to an embodiment, the lens may be configured to direct an ultrasound signal towards the region of interest of the subject. For example, the lens may be silicone, epoxy, rubber, or the like. According to an embodiment, the acoustic matching layer may be configured to facilitate matching of an impedance differential that may exist between the relatively high impedance transducer elements and the relatively low impedance subject. For example, the acoustic matching layer may be graphite, plastic, resin, or the like. According to an embodiment, the transducer elements 308, respectively, may be configured to receive an element specific transmit signal, transform the element specific transmit signal to an ultrasound signal, and transmit the ultrasound signal towards a region of interest. Additionally, or alternatively, the transducer elements 308 may be configured to receive an echo signal reflected by or backscattered from the region of interest, transform the echo signal to an electrical signal, and transmit the electrical signal. For example, the transducer elements 308 may be piezoelectric materials, such as Pb(Mg_{1/3}Nb_{2/3})O₃—PbTiO₃ (“PMN-PT”), Pb(In_{1/2}Nb_{1/2})O₃—Pb (Mg_{1/3}Nb_{2/3})O₃—PbTiO₃ (“PIN-PMN-PT”), Pb(ZrTi) (“PZT”), or the like. According to an embodiment, the acoustic dematching layer may be configured to decrease insertion losses and enhance a frequency bandwidth of the transducer elements 308. For example, the acoustic dematching layer may be tungsten carbide, silicon carbide, or the like. According to an embodiment, the backing layer may be configured to attenuate ultrasound signals directed from the transducer elements 308 in a direction opposite to the subject, and attenuate ultrasound signals deflected by a housing of the ultrasound probe 302. For example, the backing layer may be an epoxy, a metal, or the like.

The transmit beamformer 304 may be configured to apply delay times to electrical signals provided to the elements 308 to focus corresponding ultrasound signals at the region of interest. The transmitter 306 may be configured to transmit electrical signals to the elements 308 to drive the elements 308 to emit ultrasound signals towards the region of interest. The elements 308 may be configured to receive the electrical signals from the transmitter 306, convert the electrical signals into ultrasound signals, and emit the ultrasound signals towards the region of interest. The elements 308 may be configured to receive echo ultrasound signals that are back-scattered by the region of interest, convert the echo ultrasound signals into electrical signals, and provide the electrical signals to the receiver 310. The receiver 310 may be configured to receive electrical signals from the elements 308, and provide the electrical signals to the receive beamformer 312. The receive beamformer 312 may apply delay times to the electrical signals received from the elements 308.

The user input device 314 may be configured to receive a user input, and provide the user input to the processor 316. For example, the user input device 314 may be a touch screen display, a keyboard, a keypad, a mouse, a button, a switch, a microphone, or the like. Additionally, or alternatively, the user input device 314 may be configured to sense information. For example, the user input device 314 may sense information from an electro-magnetic positioning system, an inertial measurement system, an accelerometer, a gyroscope, an actuator, or the like.

The processor 316 may be configured to perform the operations as described herein. For example, the processor 316 may be a CPU, a GPU, an APU, a microprocessor, a microcontroller, a DSP, an FPGA, an ASIC, or another type of processing component. The processor 316 may be implemented in hardware, firmware, or a combination of hardware and software. The processor 316 may include one or more processors 316 configured to perform the operations described herein. For example, a single processor 316 may be configured to perform all of the operations described herein. Alternatively, multiple processors 316, collectively, may be configured to perform all of the operations described herein, and each of the multiple processors 316 may be configured to perform a subset of the operations described herein. For example, a first processor 316 may perform a first subset of the operations described herein, a second processor 316 may be configured to perform a second subset of the operations described herein, etc.

The processor 316 may be configured to control the ultrasound probe 302 to receive ultrasound data. The processor 316 may be configured to control which of the elements 308 are active, and control the shape of a beam emitted from the ultrasound probe 302. The processor 316 may generate ultrasound images for display. For example, the processor 316 may generate B-mode images, color Doppler images, anatomical M-mode images, color M-mode images, or the like. The ultrasound images may be 3D images, 2D images, single plane images, bi-plane images, three-plane images, multi-plane images, or the like. The ultrasound images may correspond to various anatomical planes (e.g., sagittal, coronal, and transverse) of the region of interest.

The display 318 may be configured to display information. For example, the display 318 may be a monitor, an LED display, a cathode ray tube, a projector display, a touchscreen, tablet computer, mobile phone, or the like. The display 318 may display ultrasound images based on the ultrasound data in real-time. For example, the display 318 may display the ultrasound images within one second, two seconds, five seconds, etc., of the ultrasound data being received by the ultrasound probe 302.

The memory 320 may be configured to store information and/or instructions for use by the processor 316. The memory 320 may be a non-transitory computer-readable medium. For example, the memory 320 may be a random access memory (RAM), a read only memory (ROM), and/or another type of dynamic or static storage device (e.g., a flash memory, a magnetic memory, and/or an optical memory) that stores information and/or instructions for use by the processor 316. The memory 320 may be configured to store instructions that, when executed by the processor 316, cause the processor 316 to perform the operations described herein.

The communication interface 322 may be configured to enable the processor 316 to communicate with other systems, such as via a wired connection, a wireless connection, or a combination of wired and wireless connections. For example, the communication interface 322 may include an Ethernet interface, an optical interface, a coaxial interface, an infrared interface, an RF interface, a USB interface, a Wi-Fi interface, a cellular network interface, or the like.

The number and arrangement of the components of the ultrasound system 120 shown in FIG. 3 are provided as an example. In practice, the ultrasound system 120 may include additional components, fewer components, different components, or differently arranged components than those shown in FIG. 3. Additionally, or alternatively, a set of components (e.g., one or more components) of the ultrasound system 120 may perform one or more functions described as being performed by another set of components of the ultrasound system 120.

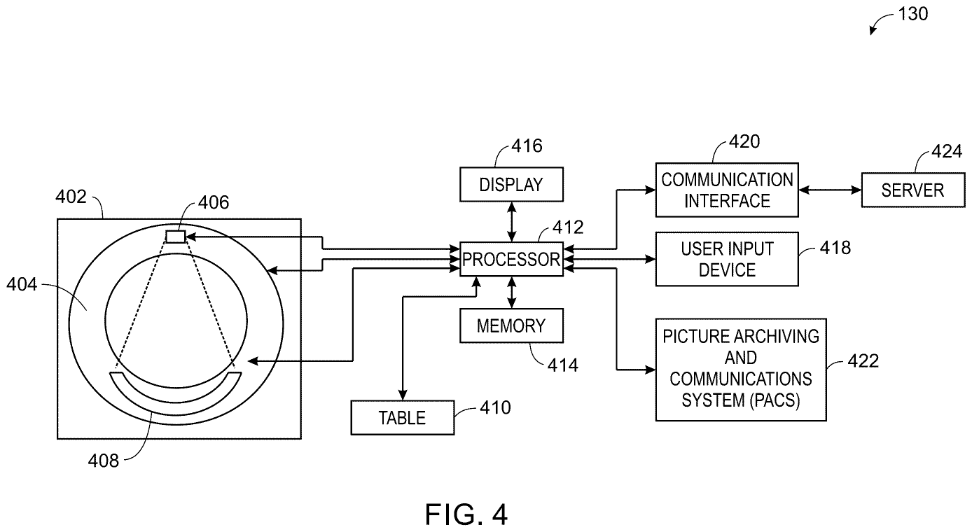

FIG. 4 is a diagram of an example preoperative imaging system for receiving preoperative imaging data of the region of interest of the anatomical feature of the heart of the subject. As shown in FIG. 4, the preoperative imaging system 130 may include a gantry 402, a rotational frame 404, an X-ray source 406, an X-ray detector 408, a table 410, a processor 412, a memory 414, a display 416, a user input device 418, a communication interface 420, a picture archiving and communications system (PACS) 422, and a server 424.

The processor 412 may be configured to control operations of the preoperative imaging system 130. For example, the processor 412 may be a CPU, a GPU, an APU, a microprocessor, a microcontroller, a DSP, an FPGA, an ASIC, or the like. The processor 412 may be implemented in hardware, firmware, or a combination of hardware and software. The processor 412 may include one or more processors 412 configured to perform the operations described herein. For example, a single processor 412 may be configured to perform all of the operations described herein. Alternatively, multiple processors 412, collectively, may be configured to perform all of the operations described herein, and each of the multiple processors 412 may be configured to perform a subset of the operations described herein. For example, a first processor 412 may perform a first subset of the operations described herein, a second processor 412 may be configured to perform a second subset of the operations described herein, etc.

The processor 412 may be configured to control the gantry 402, movement of the rotational frame 404, the X-ray source 406, the X-ray detector 408, and movement of the table 410.

The memory 414 may be configured to store information and/or instructions for use by the processor 412. The memory 414 may be a non-transitory computer-readable medium. For example, the memory 414 may be a RAM, a ROM, a flash memory, a magnetic memory, an optical memory, or the like. The memory 414 may be configured to store instructions that, when executed by the processor 412, cause the processor 412 to perform the operations described herein.

The display 416 may be configured to display information. For example, the display 416 may be a monitor, an LED display, a cathode ray tube, a projector display, a touchscreen, tablet computer, mobile phone, or the like.

The user input device 418 may be configured to receive a user input, and provide the user input to the processor 412. For example, the user input device 418 may be a touch screen display, a keyboard, a keypad, a mouse, a button, a switch, a microphone, or the like. Additionally, or alternatively, the user input device 418 may be configured to sense information. For example, the user input device 418 may sense information from an electro-magnetic positioning system, an inertial measurement system, an accelerometer, a gyroscope, an actuator, or the like.

The communication interface 420 may be configured to enable the processor 412 to communicate with other devices, such as via a wired connection, a wireless connection, or a combination of wired and wireless connections. For example, the communication interface 420 may include an Ethernet interface, an optical interface, a coaxial interface, an infrared interface, an RF interface, a USB interface, a Wi-Fi interface, a cellular network interface, or the like. The PACS 422 may be configured to communicate with external systems and/or networks to permit users at various locations to access the medical image. The server 424 may be configured to store one or more models as described herein. For example, the server 424 may be an on-premises server, a cloud server, a virtual machine, or the like.

The number and arrangement of the components of the preoperative imaging system 130 shown in FIG. 4 are provided as an example. In practice, the preoperative imaging system 130 may include additional components, fewer components, different components, or differently arranged components than those shown in FIG. 4. Additionally, or alternatively, a set of components (e.g., one or more components) of the preoperative imaging system 130 may perform one or more functions described as being performed by another set of components of the preoperative imaging system 130.

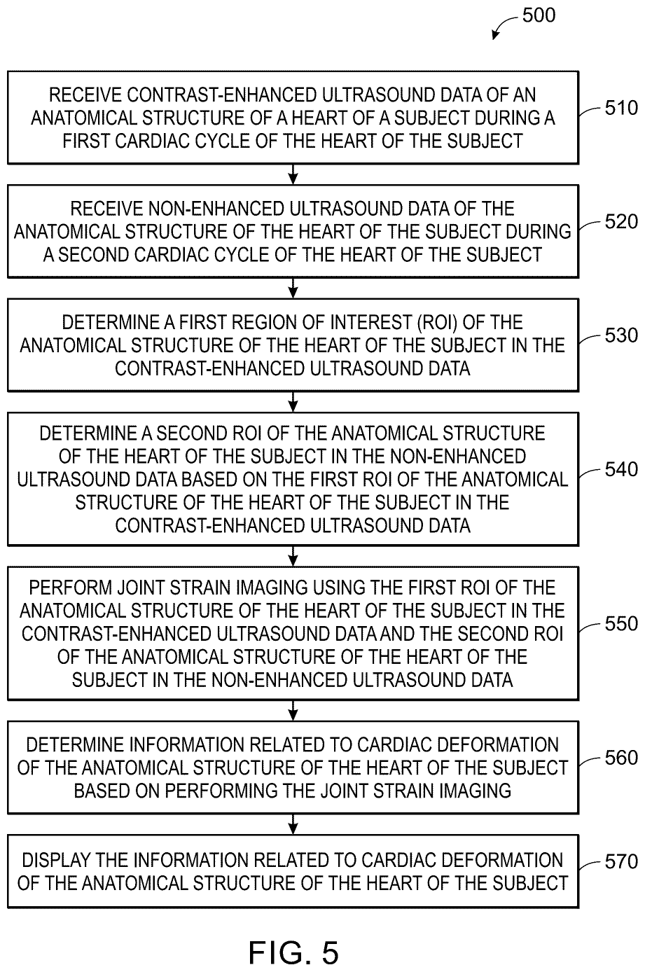

FIG. 5 is a flowchart of an example process 500 for performing joint strain imaging using contrast-enhanced ultrasound data and non-enhanced ultrasound data.

As shown in FIG. 5, the process 500 may include receiving contrast-enhanced ultrasound data of an anatomical structure of a heart of a subject during a first cardiac cycle of the heart of the subject (operation 510). For example, the strain imaging system 110 may receive contrast-enhanced ultrasound data of an anatomical structure of a heart of a subject during a first cardiac cycle of the heart of the subject.

According to an embodiment, the anatomical structure of the heart may be the left atrium, the left ventricle, the right atrium, the right ventricle, the mitral valve, the aortic valve, the pulmonary valve, the tricuspid valve, or the like. The subject may be a patient, an animal, a phantom, or the like. Although the embodiments herein are described in connection with cardiac structures, it should be understood that the embodiments herein are applicable to non-cardiac structures.

According to an embodiment, the contrast-enhanced ultrasound data may be acquired during a first cardiac cycle of the heart of the subject. For example, the first cardiac cycle may be a specific cardiac cycle of the heart of the subject. According to an embodiment, the contrast-enhanced ultrasound data may be acquired at a first frame rate. For example, the first frame rate may be any frame rate for acquisition of contrast-enhanced ultrasound data. As examples, the first frame rate may be fifty frames per second, fifty-five frames per second, sixty frames per second, or the like.

According to an embodiment, the contrast-enhanced ultrasound data may be acquired using contrast-enhanced echocardiography. For example, the ultrasound system 120 may acquire the contrast-enhanced ultrasound data using contrast-enhanced echocardiography, and provide the contrast-enhanced ultrasound data to the strain imaging system 110. It should be understood that, in some embodiments, the strain imaging system 110 and the ultrasound system 120 may be the same underlying system.

As further shown in FIG. 5, the process 500 may include receiving non-enhanced ultrasound data of the anatomical structure of the heart of the subject during a second cardiac cycle of the heart of the subject (operation 520). For example, the strain imaging system 110 may receive non-enhanced ultrasound data of the anatomical structure of the heart of the subject during a second cardiac cycle of the heart of the subject.

According to an embodiment, the non-enhanced ultrasound data may be acquired during a second cardiac cycle of the heart of the subject. For example, the second cardiac cycle may be a specific cardiac cycle of the heart of the subject. The second cardiac cycle may be a subsequent cardiac cycle as compared to the first cardiac cycle. For example, the second cardiac cycle may be the immediately subsequent cardiac cycle as compared to the first cardiac cycle. Alternatively, one or more intervening cardiac cycles may exist between the first cardiac cycle and the second cardiac cycle. Switching from contrast acquisition to non-enhanced acquisition may destroy the contrast medium due to the higher mechanical index in the non-enhanced imaging mode. Therefore, the strain imaging system 110 may set the second cycle to be suitable for strain imaging. To avoid any potential residual effects, the strain imaging system 110 may refrain from acquiring the non-enhanced ultrasound data until multiple cardiac cycles have occurred before acquiring the non-enhanced ultrasound data. Alternatively, the second cardiac cycle may be the same cardiac cycle as the first cardiac cycle. Restated, the strain imaging system 110 may acquire contrast-enhanced ultrasound data of an anatomical structure of a heart of a subject during a particular cardiac cycle of the heart of the subject using contrast-enhanced echocardiography, and may acquire non-enhanced ultrasound data of the anatomical structure of the heart of the subject during the same particular cardiac cycle of the heart of the subject using echocardiography. This technique may eliminate the time offset between the contrast-enhanced ultrasound data and the non-enhanced ultrasound data. Further, this technique may remove the need for interpolation.

According to an embodiment, the non-enhanced ultrasound data may be acquired at a second frame rate. For example, the second frame rate may be any frame rate for acquisition of non-enhanced ultrasound data, and may be greater than the first frame rate. As examples, the second frame rate may be one hundred and fifty frames per second, one hundred and fifty-five frames per second, one hundred and sixty frames per second, or the like. Further, as another example, if the first frame rate is fifty frames per second, then the second frame rate may be one hundred and fifty frames per second.

According to an embodiment, the non-enhanced ultrasound data may be acquired using non-enhanced echocardiography. For example, the ultrasound system 120 may acquire the non-enhanced ultrasound data using non-enhanced echocardiography (e.g., B-mode imaging), and provide the non-enhanced ultrasound data to the strain imaging system 110.

As further shown in FIG. 5, the process 500 may include determining a first region of interest (ROI) of the anatomical structure of the heart of the subject in the contrast-enhanced ultrasound data (operation 530). For example, the strain imaging system 110 may determine a first ROI of the anatomical structure of the heart of the subject in the contrast-enhanced ultrasound data.

According to an embodiment, the strain imaging system 110 may determine the first ROI of the anatomical structure of the heart of the subject in the contrast-enhanced ultrasound data using an image processing technique. For example, the image processing technique may be a segmentation technique, a pattern matching technique, a feature extraction technique, an image analysis technique, an edge detection technique, an image registration technique, or the like. In this case, the strain imaging system 110 may analyze the contrast-enhanced ultrasound data using the image processing technique, and determine the first ROI of the anatomical structure of the heart of the subject based on the analysis.

According to an embodiment, the strain imaging system 110 may determine the first ROI of the anatomical structure of the heart of the subject in the contrast-enhanced ultrasound data using an AI model. The AI model may be a convolutional neural network (CNN) model, a residual neural network, a random forest model, a decision tree model, an artificial neural network (ANN), a Naïve Bayes model, a decision tree, a recurrent neural network (RNN), a logistic regression model, a support vector machine, or the like. Additionally, or alternatively, the AI model may be a segmentation model, such as an edge-based segmentation model, a clustering-based segmentation model, a neural network-based segmentation model, a region-based segmentation model, or the like. In this case, the strain imaging system 110 may input the contrast-enhanced ultrasound data into the AI model, and determine the first based on an output of the AI model.

According to an embodiment, the strain imaging system 110 may determine the first ROI of the anatomical structure of the heart of the subject in the contrast-enhanced ultrasound data using preoperative imaging data of the anatomical structure of the heart of the subject acquired by the preoperative imaging system 130. For example, the strain imaging system 110 may register the contrast-enhanced ultrasound data with a preoperative image, of a preoperative imaging dataset, acquired by the preoperative imaging system 130 that identifies the first ROI of the anatomical structure of the heart, and determine the first ROI of the anatomical structure of the heart based on registering the contrast-enhanced ultrasound data with a preoperative image. In this case, the preoperative imaging data may identify the one or more flow regions. In an embodiment, the preoperative imaging data may be automatically analyzed to determine the first ROI of the anatomical structure of the heart. Alternatively, the preoperative imaging data may be manually labelled with the first ROI of the anatomical structure of the heart.

According to an embodiment, the strain imaging system 110 may determine the first ROI of the anatomical structure of the heart of the subject in the contrast-enhanced ultrasound data based on a user input. For example, a user of the strain imaging system 110 may interact with a user input device to select the first ROI of the anatomical structure of the heart of the subject. The strain imaging system 110 may determine the first ROI of the anatomical structure of the heart of the subject based on the selection.

According to an embodiment, the strain imaging system 110 may segment the first ROI of the anatomical structure into a set of myocardial segments. For example, the strain imaging system 110 may segment the first ROI into a set of n myocardial segments. Additionally, or alternatively, the strain imaging system 110 may delineate a set of tracking points in the first ROI for strain imaging.

According to an embodiment, the strain imaging system 110 may determine the first ROI for each frame of the contrast-enhanced ultrasound data. For example, the strain imaging system 110 may receive n frames of contrast-enhanced ultrasound data, and may determine the first ROI in each of the n frames of the contrast-enhanced ultrasound data.

As further shown in FIG. 5, the process 500 may include determining a second ROI of the anatomical structure of the heart of the subject in the non-enhanced ultrasound data based on the first ROI of the anatomical structure of the heart of the subject in the contrast-enhanced ultrasound data (operation 540). For example, the strain imaging system 110 may determine a second ROI of the anatomical structure in the heart of the subject in the non-enhanced ultrasound data based on the first ROI of the anatomical structure of the heart of the subject in the contrast-enhanced ultrasound data.

According to an embodiment, the strain imaging system 110 may receive m frames of non-enhanced ultrasound data. The m frames may be the same number of the n frames of the contrast-enhanced ultrasound data. Alternatively, the m frames may be a different number of frames as compared to the contrast-enhanced ultrasound data. For example, the m frames may be a greater number of frames than the n frames, or may be fewer frames than the n frames.

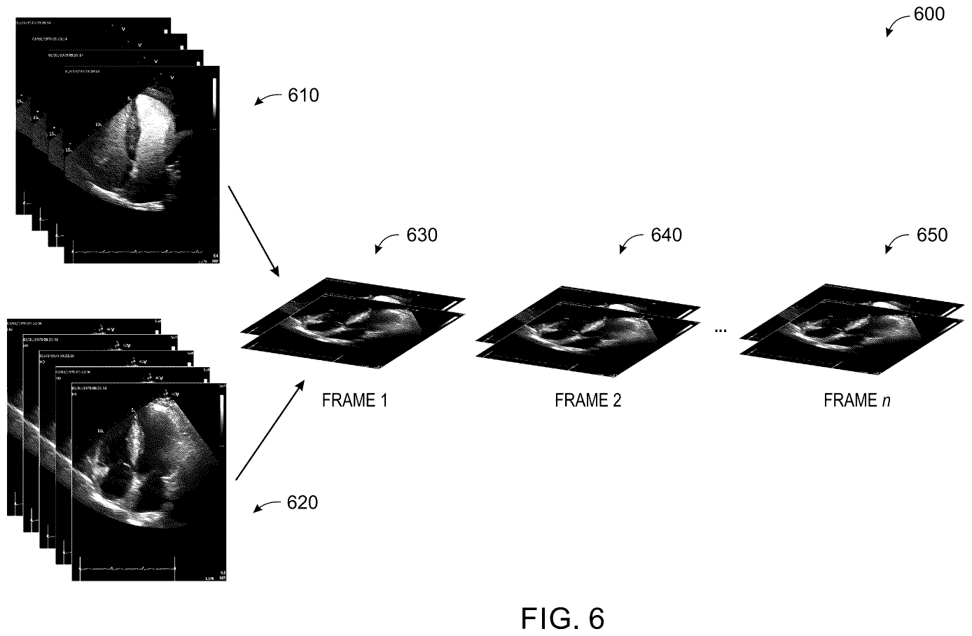

The strain imaging system 110 may determine a frame of the non-enhanced ultrasound data that corresponds to a frame of the contrast-enhanced ultrasound data. FIG. 6 is a diagram 600 of contrast-enhanced ultrasound data and non-enhanced ultrasound data. As shown in FIG. 6, the strain imaging system 110 may receive contrast-enhanced ultrasound data 610 and non-enhanced ultrasound data 620. Further, the strain imaging system 110 may determine pairs of frames that correspond to each other. For example, as shown by reference number 630, the strain imaging system 110 may determine a first frame of the contrast-enhanced ultrasound data 610 that corresponds to a first frame of the non-enhanced ultrasound data 620. Further, as shown by reference number 640, the strain imaging system 110 may determine a second frame of the contrast-enhanced ultrasound data 610 that corresponds to a second frame of the non-enhanced ultrasound data 620. Further still, as shown by reference number 650, the strain imaging system 110 may determine an n-th frame of the contrast-enhanced ultrasound data 610 that corresponds to an n-th frame of the non-enhanced ultrasound data 620.

According to an embodiment, the strain imaging system 110 may determine a frame of the non-enhanced ultrasound data that corresponds to a frame of the contrast-enhanced ultrasound data based on time stamps of the frames that identify positions of the frames in the cardiac cycle. Additionally, or alternatively, the strain imaging system 110 may determine a frame of the non-enhanced ultrasound data that corresponds to a frame of the contrast-enhanced ultrasound data based on an image processing technique. For example, the strain imaging system 110 may determine a frame of the non-enhanced ultrasound data that corresponds to a frame of the contrast-enhanced ultrasound data based on the frames corresponding to a same portion of the cardiac cycle, depicting similar anatomical structures in similar anatomical positions, etc. Additionally, or alternatively, the strain imaging system 110 may generate one or more additional frames using an interpolation technique, or the like, to determine a frame of the non-enhanced ultrasound data that corresponds to a frame of the contrast-enhanced ultrasound data.

In some situations, there might be a frame rate mismatch between the contrast-enhanced acquisitions and the non-enhanced acquisitions. That is, the non-enhanced acquisition may have a greater frame rate than as compared to the contrast-enhanced acquisition. Therefore, the exact corresponding frame selected from the contrast-enhanced ultrasound data may not appear in the non-enhanced ultrasound data. In this case, the strain imaging system 110 may determine the two nearest neighboring frames in the non-enhanced ultrasound data, and perform frame interpolation for the exact corresponding time. Additionally, the accuracy of the strain imaging system 110 might not be compromised if there is a slight frame time mismatch between the corresponding contrast-enhanced ultrasound data and the non-enhanced ultrasound data because motion can be assumed to be linear over short intervals.

According to an embodiment, the strain imaging system 110 may determine the second ROI of the anatomical structure in the heart of the subject in the non-enhanced ultrasound data based on the first ROI of the anatomical structure of the heart of the subject in the contrast-enhanced ultrasound data. For example, the strain imaging system 110 may determine a position of the second ROI of the anatomical structure in the heart of the subject in the non-enhanced ultrasound data based on a position of the first ROI of the anatomical structure of the heart of the subject in the contrast-enhanced ultrasound data. As a particular example, the strain imaging system 110 may overlay the first ROI in the non-enhanced ultrasound data to determine the second ROI in the non-enhanced ultrasound data.

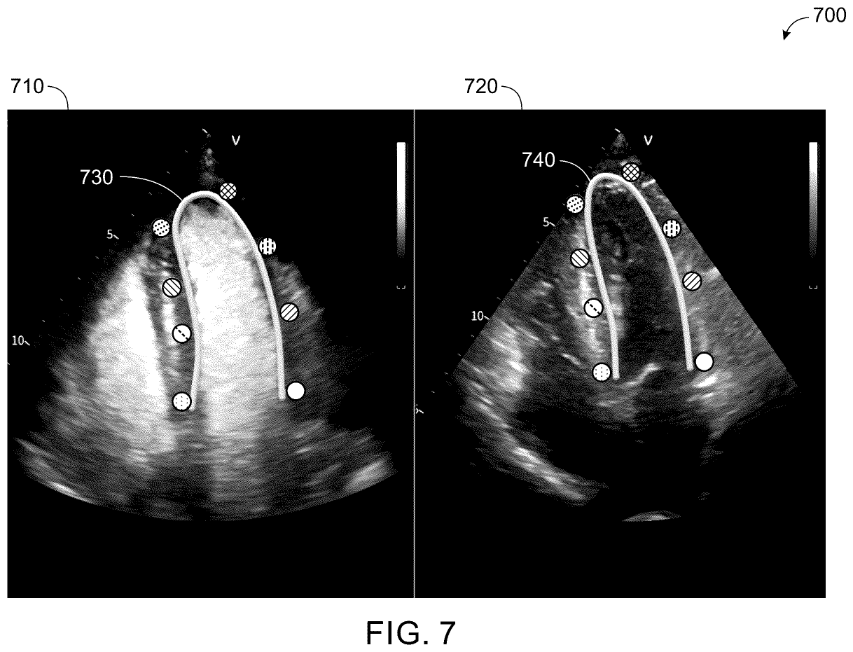

FIG. 7 is a diagram 700 of a first ROI in contrast-enhanced ultrasound data and a second ROI in non-enhanced ultrasound data. For example, as shown in FIG. 7, the strain imaging system 110 may receive a first frame of contrast-enhanced ultrasound data 710 and a first frame of non-enhanced ultrasound data 720. The strain imaging system 110 may determine a first ROI 730 in the contrast-enhanced ultrasound data 710, and may determine a second ROI 740 in the non-enhanced ultrasound data 720 based on the first ROI 730.

As further shown in FIG. 5, the process 500 may include performing joint strain imaging using the first ROI of the anatomical structure of the heart of the subject in the contrast-enhanced ultrasound data and the second ROI of the anatomical structure of the heart of the subject in the non-enhanced ultrasound data (operation 550).

To perform joint strain imaging, and according to an embodiment, the strain imaging system 110 may segment the first ROI of the anatomical structure of the heart of the subject in the contrast-enhanced ultrasound data into a set of myocardial segments, and may segment the second ROI of the anatomical structure of the heart of the subject in the non-enhanced ultrasound data into a set of myocardial segments. Further, the strain imaging system 110 may track the respective segments in the contrast-enhanced ultrasound data and the non-enhanced ultrasound data over time using strain imaging. Further still, the strain imaging system 110 may determine respective strain values of the set of myocardial segments over time based on tracking the respective segments using strain imaging. For example, the strain imaging system 110 may determine a strain value based on a starting length of a segment and a final length of the segment. As an example, if the starting length of the segment is “10” and the final length of the segment is “8,” then the strain imaging system 110 may determine a strain value of “−20%.” As another example, if the starting length of the segment is “8” and the final length of the segment is “10,” then the strain imaging system 110 may determine a strain value of “20%.” The strain imaging system 110 may generate respective strain curves for the set of myocardial segments.

The strain imaging system 110 may perform joint strain imaging by determining a common motion vector that maximizes, or improves, image similarities in the contrast-enhanced ultrasound data and the non-enhanced ultrasound data for each myocardial segment and/or tracking point. For example, the strain imaging system 110 may use the following cost function to determine a common motion vector that maximizes, or improves, image similarities in the contrast-enhanced ultrasound data and the non-enhanced ultrasound data for each myocardial segment and/or tracking point:

$$Cost ( R , C ) = \frac{1}{{N}^{2}} ( {\sum }_{i} {\sum }_{j} α \vert {C}_{ij}^{Contrast} - {R}_{ij}^{Contrast} \vert + β \vert {C}_{ij}^{Bmode} - {R}_{ij}^{Bmode} \vert )$$

As shown above, “C” is a kernel from a current frame, and “R” is a kernel from a reference frame. Further, “N” is the number of image samples in each kernel. Further, “α” and “β” are weighting parameters. The sum of the weighting parameter “α” the weighting parameter “β” may be equal to 1. The strain imaging system 110 may determine the motion vector that both of the contrast-enhanced ultrasound data and the non-enhanced ultrasound data indicate is the correct motion vector for a given speckle. This approach enhances the reliability of strain imaging by incorporating a majority vote. Since the underlying cardiac motion is independent of the acquisition mode, corresponding points from the contrast-enhanced ultrasound data and non-enhanced ultrasound data should exhibit the same, or substantially similar, motion. The weighting parameters may specify how much trust should be placed on each term (contrast-enhanced ultrasound data or non-enhanced ultrasound data). The strain imaging system 110 may be configured with predetermined information that sets the values of the weighting parameters for each tracking point. The strain imaging system 110 may assign a higher weight to a tracking point (kernel) that contains a strong feature that is suitable for feature tracking. In contrast, the strain imaging system 110 may assign a lower weight if the kernel includes a noise-like structure that reduces the confidence of feature tracking. Examples of such regions include the lateral wall near the apex in non-enhanced ultrasound data, and the mitral valve hinge point in contrast-enhanced ultrasound data. If the feature strength of both the contrast-enhanced ultrasound data and the non-enhanced ultrasound data is comparable, the strain imaging system 110 may assign substantially equal weights (e.g., 0.5).

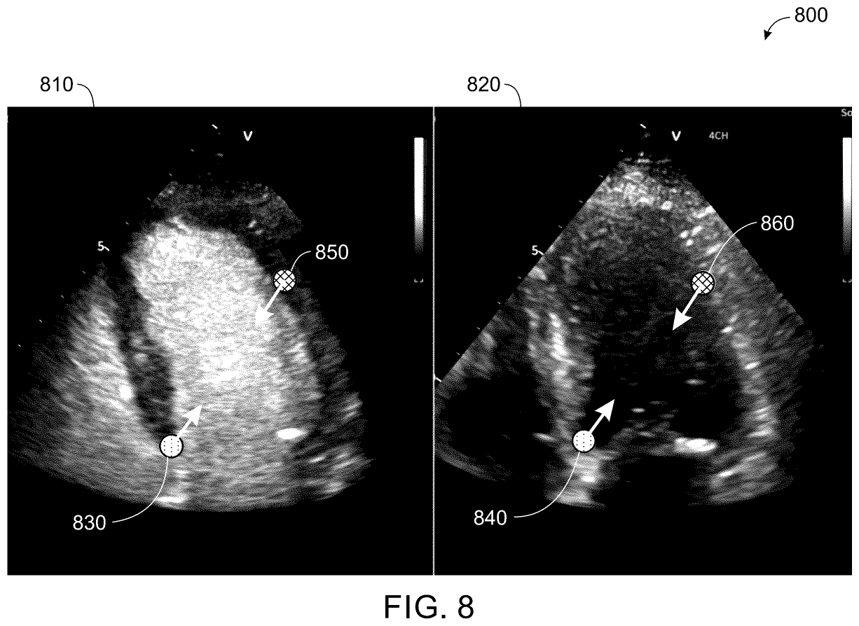

FIG. 8 is a diagram 800 of tracking points in the contrast-enhanced ultrasound data and the non-enhanced ultrasound data. For example, the strain imaging system 110 may receive a first frame of contrast-enhanced ultrasound data 810 and a first frame of non-enhanced ultrasound data 820. Further, the strain imaging system 110 may determine a first tracking point 830 in the contrast-enhanced ultrasound data 810 and a first tracking point 840 in the non-enhanced ultrasound data 820. Further, the strain imaging system 110 may determine a second tracking point 850 in the contrast-enhanced ultrasound data 810 and a second tracking point 860 in the non-enhanced ultrasound data 820. The strain imaging system 110 may determine a common motion vector that maximizes, or improves, image similarities in the contrast-enhanced ultrasound data 810 and the non-enhanced ultrasound data 820 for the first tracking point 830 in the contrast-enhanced ultrasound data 810 and the first tracking point 840 in the non-enhanced ultrasound data 820. Further, the strain imaging system 110 may determine a common motion vector that maximizes, or improves, image similarities in the contrast-enhanced ultrasound data 810 and the non-enhanced ultrasound data 820 for the second tracking point 850 in the contrast-enhanced ultrasound data 810 and the second tracking point 860 in the non-enhanced ultrasound data 820.

The true definition of the endocardial border in non-enhanced ultrasound data, especially near the apex and on the lateral wall may be ambiguous due to a weak signal. This ambiguity may lead to an overestimation of strain in apical segments because the ROI is dragged down, and geometric effects play an important role. Utilizing the first ROI that is determined from the contrast-enhanced ultrasound data on subsequent non-enhanced ultrasound data may result in a more accurate definition of segment length and, consequently, more robust strain values.

The strain imaging system 110 may detect movement of the ultrasound probe 302 and/or movement of the subject from the first cardiac cycle during which the contrast-enhanced ultrasound data is acquired to the second cardiac cycle during which the non-enhanced ultrasound data is acquired. For example, the strain imaging system 100 may compare the contrast-enhanced ultrasound data and non-enhanced ultrasound data that include the same timestamps and/or that correspond to a same portion of the cardiac cycle. As a particular example, the strain imaging system 110 may compare the contrast-enhanced ultrasound data and non-enhanced ultrasound data around the valve hinge points. If there is motion, the strain imaging system 110 may compensate for the motion and remove the motion from the estimated displacements.

As further shown in FIG. 5, the process 500 may include determining information related to cardiac deformation of the anatomical structure of the heart of the subject based on performing the joint strain imaging (operation 560). For example, the strain imaging system 110 may determine information related to cardiac deformation of the anatomical feature of the heart of the subject based on performing the joint strain imaging.

According to an embodiment, the information related to cardiac deformation of the region of interest of the heart of the subject may be strain values of myocardial segments of the heart, amounts of time to reach maximum strain values of the respective myocardial segments, particular myocardial segments that are associated with amounts of time that are greater than or less than respective thresholds, a mechanical dispersion value, a cardiac mechanical dyssynchrony parameter, or the like. Additionally, or alternatively, the information related to cardiac deformation of the region of interest heart of the subject may include an end-systolic strain that corresponds to a strain value at end-systole, peak systolic strain that corresponds to a peak strain value during systole, positive peak systolic strain that corresponds to a local myocardial stretching, a peak strain that corresponds to a peak strain value during the entire cardiac cycle, or the like. Additionally, or alternatively, the information related to cardiac deformation of the region of interest of the heart of the subject may be a velocity, a displacement, a strain rate, or the like, of a myocardial segment.

As further shown in FIG. 5, the process 500 may include displaying the information related to cardiac deformation of the anatomical structure of the heart of the subject (operation 570). For example, the strain imaging system 110 may display the information related to cardiac deformation of the anatomical feature of the heart, such as the mechanical dispersion value, strain values of the set of segments, amounts of time to reach maximum strain values of the respective segments, particular segments that are associated with amounts of time that are greater than or less than respective thresholds, or the like.

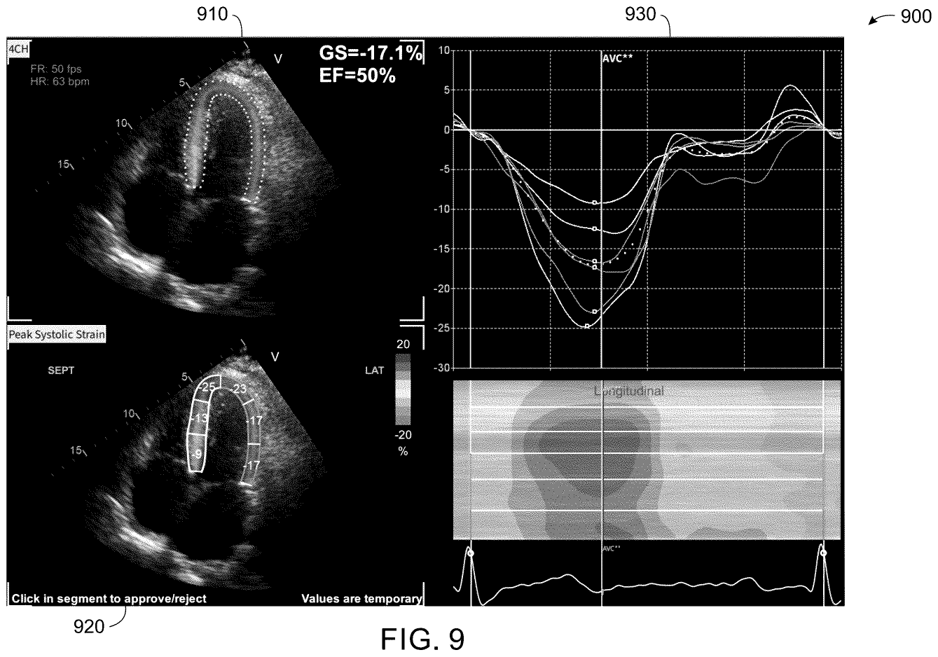

FIG. 9 is a diagram 900 of an example user interface for displaying the information related to cardiac deformation of the anatomical structure of the heart of the subject. For example, as shown, the strain imaging system 110 may display a non-enhanced ultrasound image 910 that depicts the second ROI and a delineation of the second ROI. Further, the strain imaging system 110 may display a non-enhanced ultrasound image 920 that depicts the second ROI, a delineation of the second ROI, and a delineation of various segments of the second ROI. Further, as shown, the strain imaging system 110 may display strain curves 930 for the set of segments.

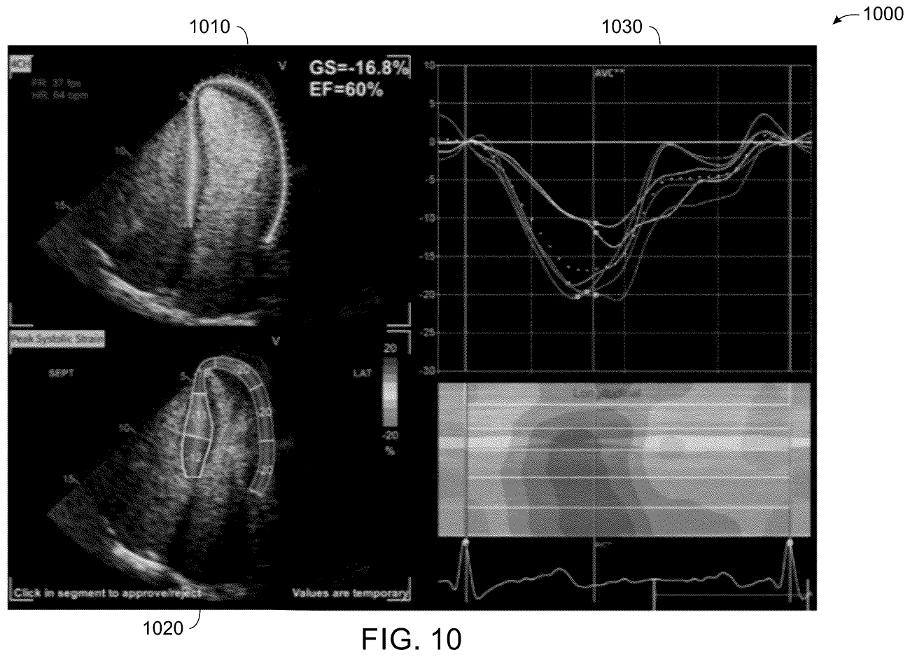

FIG. 10 is a diagram 1000 of an example user interface for displaying the information related to cardiac deformation of the anatomical structure of the heart of the subject. For example, as shown, the strain imaging system 110 may display a contrast-enhanced ultrasound image 1010 that depicts the first ROI and a delineation of the first ROI. Further, the strain imaging system 110 may display a contrast-enhanced ultrasound image 1020 that depicts the first ROI, a delineation of the first ROI, and a delineation of various segments of the first ROI. Further, as shown, the strain imaging system 110 may display strain curves 1030 for the set of segments.

Because the non-enhanced ultrasound data and the contrast-enhanced ultrasound data are used to estimate underlying cardiac motion, the estimated ROI is the same for both the non-enhanced ultrasound data and the contrast-enhanced ultrasound data. The strain imaging system 110 may providing a toggle option on the user interface with non-enhanced ultrasound data and contrast-enhanced ultrasound data selection. If contrast-enhanced ultrasound data is selected, the strain imaging system 110 may display the contrast-enhanced ultrasound data and may overlay the delineation of the first ROI on the contrast-enhanced ultrasound data. Similarly, if non-enhanced ultrasound data is selected, the strain imaging system 110 may display the non-enhanced ultrasound data and may overlay the delineation of the second ROI on the contrast-enhanced ultrasound data. This option enables the user to inspect both border tracking (e.g., contrast) and speckle tracking (e.g., B-mode) simultaneously.

According to an embodiment, the strain imaging system 110 may use one or more AI models. The one or more AI models may be associated with a training phase, a deployment phase, and a monitoring phase. In the training phase, the strain imaging system 110 may receive and process training data to generate a trained model. The training data may be generated, received, or otherwise obtained from internal and/or external resources.

Generally, the trained model may include a set of variables (e.g., nodes, neurons, filters, or the like) that are tuned (e.g., weighted, biased, or the like) to different values via the application of the training data. According to an embodiment, the training process may employ supervised, unsupervised, semi-supervised, and/or reinforcement learning processes to train the model. According to an embodiment, a portion of the training data may be withheld during training and/or used to validate the trained model.

For supervised learning processes, the training data may include labels or scores that may facilitate the training process by providing a ground truth. For example, the labels or scores may indicate an output of the model. Training may proceed by feeding a training dataset including the training data into the model. The model may have variables set at initialized values (e.g., at random, based on Gaussian noise, based on pre-trained values, or the like). The model may generate an output based on the training dataset being input to the model. The output may be compared with the corresponding label or score (e.g., the ground truth) indicating the known output, which may then be back-propagated through the model to adjust the values of the variables. This process may be repeated for a plurality of samples at least until a determined loss or error is below a predefined threshold. According to an embodiment, some of the training data may be withheld and used to further validate or test the trained model.

For unsupervised learning processes, the training data may not include pre-assigned labels or scores to aid the learning process. Instead, unsupervised learning processes may include clustering, classification, or the like, to identify naturally occurring patterns in the training data. As an example, the training data may be clustered into groups based on identified similarities and/or patterns. K-means clustering or K-Nearest Neighbors may also be used, which may be supervised or unsupervised. Combinations of K-Nearest Neighbors and an unsupervised cluster technique may also be used. For semi-supervised learning, a combination of training data with pre-assigned labels or scores and training data without pre-assigned labels or scores may be used to train the model.

When reinforcement learning is employed, an agent (e.g., an algorithm) may be trained to make a decision from the training data through trial and error. For example, based on making a decision, the agent may then receive feedback (e.g., a positive reward if the prediction was above a predetermined threshold), adjust its next decision to maximize the reward, and repeat until a loss function is optimized.

After being trained, the trained model may be stored and subsequently applied by the strain imaging system 110 during the deployment phase. For example, during the deployment phase, the trained model executed by the strain imaging system 110 may receive input data. During the deployment phase, the trained model may perform one or more operations as described in connection with FIG. 5.

Embodiments of the present disclosure shown in the drawings and described above are example embodiments only and are not intended to limit the scope of the appended claims, including any equivalents as included within the scope of the claims. Various modifications are possible and will be readily apparent to the skilled person in the art. It is intended that any combination of non-mutually exclusive features described herein are within the scope of the present invention. That is, features of the described embodiments can be combined with any appropriate aspect described above and optional features of any one aspect can be combined with any other appropriate aspect. Similarly, features set forth in dependent claims can be combined with non-mutually exclusive features of other dependent claims, particularly where the dependent claims depend on the same independent claim. Single claim dependencies may have been used as practice in some jurisdictions require them, but this should not be taken to mean that the features in the dependent claims are mutually exclusive.

What is claimed is:

1. A system comprising:
a lens, an acoustic matching layer, an acoustic dematching layer, and a plurality of transducer elements;
a memory configured to store instructions; and
one or more processors configured to execute the instructions to:
receive contrast-enhanced ultrasound data of an anatomical structure of a heart of a subject during a first cardiac cycle of the heart of the subject;
receive non-enhanced ultrasound data of the anatomical structure of the heart of the subject during a second cardiac cycle of the heart of the subject;
determine a first region of interest (ROI) of the anatomical structure of the heart of the subject in the contrast-enhanced ultrasound data;
determine a second ROI of the anatomical structure of the heart of the subject in the non-enhanced ultrasound data based on the first ROI of the anatomical structure of the heart of the subject in the contrast-enhanced ultrasound data;
perform joint strain imaging using the first ROI of the anatomical structure of the heart of the subject in the contrast-enhanced ultrasound data and the second ROI of the anatomical structure of the heart of the subject in the non-enhanced ultrasound data;
determine information related to cardiac deformation of the anatomical structure of the heart of the subject based on performing the joint strain imaging; and
display the information related to cardiac deformation of the anatomical structure of the heart of the subject.

2. The system of claim 1, wherein the one or more processors are further configured to:
overlay the first ROI from the contrast-enhanced ultrasound data in the non-enhanced ultrasound data to determine the second ROI of the anatomical structure of the heart of the subject in the non-enhanced ultrasound data.

3. The system of claim 1, wherein the one or more processors are further configured to:
determine a respective common motion vector for each respective tracking point in the contrast-enhanced ultrasound data and the non-enhanced ultrasound data to perform the joint strain imaging.

4. The system of claim 1, wherein the one or more processors are further configured to:
display the contrast-enhanced ultrasound data based on a user selection that selects the contrast-enhanced ultrasound data to be displayed.

5. The system of claim 1, wherein the one or more processors are further configured to:
display the non-enhanced ultrasound data based on a user selection that selects the non-enhanced ultrasound data to be displayed.

6. The system of claim 1, wherein the second cardiac cycle is immediately subsequent to the first cardiac cycle.

7. The system of claim 1, wherein one or more intervening cardiac cycles exist between the first cardiac cycle and the second cardiac cycle.

8. A method comprising:
receiving contrast-enhanced ultrasound data of an anatomical structure of a heart of a subject during a first cardiac cycle of the heart of the subject;
receiving non-enhanced ultrasound data of the anatomical structure of the heart of the subject during a second cardiac cycle of the heart of the subject;
determining a first region of interest (ROI) of the anatomical structure of the heart of the subject in the contrast-enhanced ultrasound data;
determining a second ROI of the anatomical structure of the heart of the subject in the non-enhanced ultrasound data based on the first ROI of the anatomical structure of the heart of the subject in the contrast-enhanced ultrasound data;
performing joint strain imaging using the first ROI of the anatomical structure of the heart of the subject in the contrast-enhanced ultrasound data and the second ROI of the anatomical structure of the heart of the subject in the non-enhanced ultrasound data;
determining information related to cardiac deformation of the anatomical structure of the heart of the subject based on performing the joint strain imaging; and
displaying the information related to cardiac deformation of the anatomical structure of the heart of the subject.

9. The method of claim 8, further comprising:
overlaying the first ROI from the contrast-enhanced ultrasound data in the non-enhanced ultrasound data to determine the second ROI of the anatomical structure of the heart of the subject in the non-enhanced ultrasound data.

10. The method of claim 8, further comprising:
determining a respective common motion vector for each respective tracking point in the contrast-enhanced ultrasound data and the non-enhanced ultrasound data to perform the joint strain imaging.

11. The method of claim 8, further comprising:
displaying the contrast-enhanced ultrasound data based on a user selection that selects the contrast-enhanced ultrasound data to be displayed.

12. The method of claim 8, further comprising:
displaying the non-enhanced ultrasound data based on a user selection that selects the non-enhanced ultrasound data to be displayed.

13. The method of claim 8, wherein the second cardiac cycle is immediately subsequent to the first cardiac cycle.

14. The method of claim 8, wherein one or more intervening cardiac cycles exist between the first cardiac cycle and the second cardiac cycle.

15. A non-transitory computer-readable medium storing instructions that, when executed by one or more processors, cause the one or more processors to:
receive contrast-enhanced ultrasound data of an anatomical structure of a heart of a subject during a first cardiac cycle of the heart of the subject;
receive non-enhanced ultrasound data of the anatomical structure of the heart of the subject during a second cardiac cycle of the heart of the subject;
determine a first region of interest (ROI) of the anatomical structure of the heart of the subject in the contrast-enhanced ultrasound data;
determine a second ROI of the anatomical structure of the heart of the subject in the non-enhanced ultrasound data based on the first ROI of the anatomical structure of the heart of the subject in the contrast-enhanced ultrasound data;
perform joint strain imaging using the first ROI of the anatomical structure of the heart of the subject in the contrast-enhanced ultrasound data and the second ROI of the anatomical structure of the heart of the subject in the non-enhanced ultrasound data;
determine information related to cardiac deformation of the anatomical structure of the heart of the subject based on performing the joint strain imaging; and
display the information related to cardiac deformation of the anatomical structure of the heart of the subject.

16. The non-transitory computer-readable medium of claim 15, wherein the instructions further cause the one or more processors to:
overlay the first ROI from the contrast-enhanced ultrasound data in the non-enhanced ultrasound data to determine the second ROI of the anatomical structure of the heart of the subject in the non-enhanced ultrasound data.

17. The non-transitory computer-readable medium of claim 15, wherein the instructions further cause the one or more processors to:
determine a respective common motion vector for each respective tracking point in the contrast-enhanced ultrasound data and the non-enhanced ultrasound data to perform the joint strain imaging.

18. The non-transitory computer-readable medium of claim 15, wherein the instructions further cause the one or more processors to:
display the contrast-enhanced ultrasound data based on a user selection that selects the contrast-enhanced ultrasound data to be displayed.

19. The non-transitory computer-readable medium of claim 15, wherein the instructions further cause the one or more processors to:
display the non-enhanced ultrasound data based on a user selection that selects the non-enhanced ultrasound data to be displayed.

20. The non-transitory computer-readable medium of claim 15, wherein the second cardiac cycle is immediately subsequent to the first cardiac cycle, or wherein one or more intervening cardiac cycles exist between the first cardiac cycle and the second cardiac cycle.
